(19) United States
(12) Patent Application Publication
(10) Pub. No.: US 2016/0324758 A1
(43) Pub. Date: Nov. 10, 2016

(54) COSMETIC ASSEMBLY INCLUDING A POURED PRODUCT

(71) Applicant: LVMH RECHERCHE, Saint Jean de Braye (FR)

(72) Inventor: Lily Ann Perrin, Saint Jean de Braye (FR)

(21) Appl. No.: 15/108,658

(22) PCT Filed: Dec. 12, 2014
(86) PCT No.: PCT/FR2014/053313

§ 371 Date: Jun. 28, 2016

(30) Foreign Application Priority Data
Dec. 31, 2013 (FR) 1363742

(51) Int. Cl. A61K 8/92; A45D 33/00; A45D 33/18; A45D 40/16; A61K 8/02; A61K 8/81; A61K 8/19; A61K 8/34; A61K 8/891; A61K 8/84; A61K 8/42; A61K 8/06; A61Q 17/04; A61K 8/895; A61Q 1/06; A61Q 1/10; A45D 40/24

(52) U.S. Cl. CPC A61K 8/92; A45D 40/24; A45D 33/008; A45D 33/18; A45D 40/16; A61K 8/022; A61K 8/8111; A61K 8/927; A61K 8/19; A61K 8/342; A61K 8/891; A61K 8/84; A61K 8/42; A61K 8/064; A61Q 17/04; A61K 8/895; A61Q 1/06; A61Q 1/10; A61K 8/8117; A61K 2800/24; A61K 2800/87; A61K 2800/436; A61K 2800/43

(57) ABSTRACT

A subject-matter of the invention is a cosmetic product comprising a container having at least one cavity in which is positioned an assembly of a solid inclusion part and of a fixing composition having a drop point greater than 50° C. and a mean hardness of less than 2800 g.

The invention also relates to a process for the manufacture of this product which consists in preparing the inclusion part, in depositing it on the bottom of the container and in then casting the fixing composition in order for it to surround the part and fix it to the container. This invention makes it possible to present several cosmetic products without having to resort to a compartmentalized container.

The invention relates to the field of cosmetic products, intended in particular for caring for or making up the face, which are composed of several different solid compositions. The present invention relates in particular to a cosmetic product providing several compositions of different colours and/or shapes in one and the same container.

The cosmetic product of the invention exhibits the novel feature of simultaneously comprising a composition which has been moulded or pressed and a composition which has been hot cast in the container.

### PRIOR ART

The cases used for the simultaneous presentation of several cosmetic compositions are generally flat-bottomed packagings, their function being that of presenting to the user, on any occasion, a large surface area of product in order to facilitate the choice of the colour (while avoiding having to open several different cases), in order to facilitate withdrawal using the finger or an applicator and to give an attractive appearance to the product. This surface is generally substantially flat.

The cases of the prior art are generally comprised of two parts: a lid and a container which are articulated around a hinge. The container is generally composed of several integral compartments of the case, each containing different colours.

Generally, the cases of the prior art comprise a hollow lid which can be provided with an internal mirror, and a hollow base which can be provided with an intermediate support of one or more pots which are each intended to receive a different composition. The lid and the base exhibit sidewalls substantially perpendicular to the dispensing surface, and the compositions which fill the pots are not in contact with one another in the case.

Multiple methods exist in the field of cosmetic powders for creating assemblies of colours and textures in one and the same compartment of the case.

In the field of cast cosmetics, there exists, on the other hand, a limited number of methods for creating assemblies inside one and the same case compartment. Thus, U.S. Pat. No. 6,058,942 has provided a process for the manufacture of a cosmetic product packaged in a case which consists in depositing a first solid material in the container, in pouring a second material in the liquid form into the case and then leaving it to cool in order for it to become solid. The first material has a higher melting point than that of the second material, so as not to melt when it comes into contact with the second molten material.

In the same way, Patent EP 1 325 692 puts together cast anhydrous products or pressed powders with a thermosetting resin. The resin is cast but exhibits the disadvantage of requiring a curing time of several hours. In addition, the resin cannot be used as make-up or care product.

In Patent FR 2 956 833, silicone moulds and several casting stages have been used to create multi-colours of anhydrous products in strata. The product surface accessible to the user is thus composed of just one colour. This product cannot simultaneously offer several different colours or textures to the user.

Finally, assemblies of pressed powders with cast cosmetic products have not been described.

### OBJECTIVES OF THE INVENTION

It is desired to develop cosmetic products which comprise, in one and the same packaging, indeed even in one and the same container, several colours and/or several patterns of moulded compositions or of powder cakes cut out with a laser.

It is also desired for the assembly not to use pots and to make it possible to obtain a transportable product in which the colours and the shapes are integral with one another and do not become detached over time.

On following the teaching of U.S. Pat. No. 6,058,942, the inventors have found that the second material which is cast around the first placed beforehand in the container can solidify before uniformly filling the container and before completely surrounding the first material. With a second material having a lower melting point than the first, the inventors also observed that it is not always possible to completely fill small-size interstices.

In the context of the invention, the inventors have found that it is possible to hot cast a cosmetic product around one or more cosmetic elements, having varied shapes, reliefs and colours, in order to make up a solid assembly fixed to a case. The cosmetic inclusion elements of the product of the invention are either cast shaped products or pressed powders. The inventors have found a fixing composition which matches well the inclusion shapes, matches well the shape of the container and thoroughly fills the interstices left between the inclusion shapes and the walls of the case, even when they are narrow.

The invention exhibits the advantage of using a cosmetic product to assemble and fix the cosmetic inclusion products. The whole of the assembly is thus cosmetic. Furthermore, the rate of solidification of the cosmetic product used for the fixing is of the order of a few minutes, which makes the handling much easier and the manufacturing process faster.

The invention is illustrated with reference to the Figures.

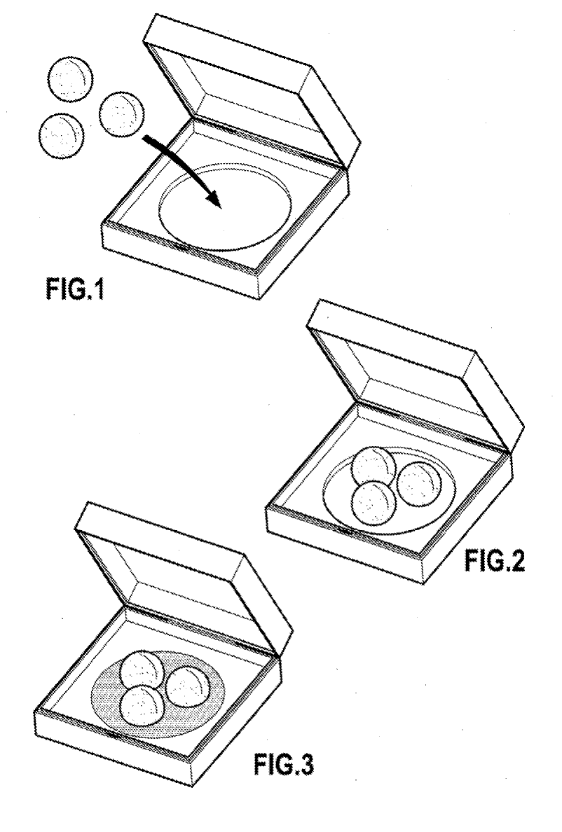

FIGS. 1 to 3 represent a view in perspective of an assembly according to the invention obtained from spherical inclusion elements which have been moulded beforehand, placed in the bottom of a container comprising a circular recess and then assembled by casting and solidification of a fixing composition comprising waxes, oils and pastes.

FIGS. 4 to 6 represent an assembly in the form of a cameo comprising an inclusion part which has been moulded and placed in a container comprising an oval recess and then immobilized by casting and solidification of a fixing composition comprising waxes, oils and pastes.

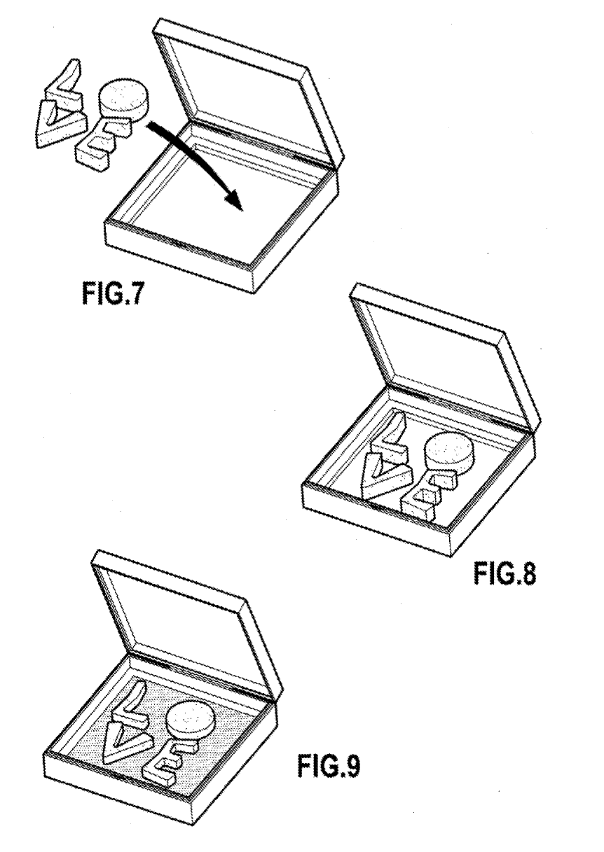

FIGS. 7 to 9 represent a view in perspective of an assembly of inclusion parts formed of pressed powders placed in the rectangular recess of a container. The inclusion parts are rendered integral by casting and solidification of a fixing composition in the form of a pearlescent transparent gel comprising oils gelled by a polymer.

### DESCRIPTION OF THE INVENTION

According to a first aspect, the invention relates to an in relief cosmetic product comprising a container having at least one cavity in which is positioned an assembly of at least one inclusion part which is solid at 25° C. and of a fixing composition which is solid at 25° C., characterized in that:
the inclusion part and the fixing composition are in contact with one another and have different compositions,
the fixing composition has a drop point greater than 50° C. and a mean hardness, measured at 25° C. using a texture analyser provided with a hemispheric probe, of less than 2800 g, and
the fixing composition comprises at least one oil and one structuring compound.

This is because it has been discovered, in the context of the present invention, that the casting of a specific product around the moulded or pressed cosmetic elements for the purpose of assembling them in a pack makes it possible to firmly fix them to one another in one and the same container, without detrimentally affecting their structure, while adhering well to the surfaces of the container, while uniformly covering the surfaces of the container and while exhibiting a relatively rapid rate of cooling.

The invention exhibits the advantage of making possible the assembling of several parts formed of cosmetic products which have been shaped independently beforehand. The product of the invention also makes possible the presentation and the unification of several textures in one and the same container, for example two cast products of different hardnesses, or a powder product and a cast product, without having recourse to the use of conventional pots which will be fixed in the case.

Finally, the product of the invention makes possible great adaptability to different shapes of cases without needing to look for a pot format which is perfectly adapted thereto or without needing to adapt the shaping formats of the cosmetic products.

The container of the product of the invention can take on any shape, such as circular or rectangular. It preferably comprises just one cavity, the shape of which is advantageously independent of that of the container and which can be rectangular, circular or oval. The container of the product of the invention is advantageously pot-free. The cavity is preferably noncompartmentalized and its bottom is flat, with the result that a liquid fills it completely when it is poured therein. In a preferred embodiment, the container comprises just one cavity and is pot-free.

In the container, the fixing composition advantageously covers the entire surface of the bottom of the cavity and preferably allows the inclusion part to show on the surface.

The product of the invention can comprise at least one inclusion part, for example at least two. The inclusion parts can advantageously be different in composition, colour and/or texture.

According to one embodiment, the inclusion part has a greater hardness than that of the fixing composition, in particular when the inclusion part predominantly comprises fatty substances, such as oils or waxes.

The inclusion parts can be anhydrous or can comprise an aqueous phase and a fatty phase.

The textures of cast anhydrous type used for the inclusion part are preferably hard textures which withstand removal from the mould and which retain well the potentially subtle shapes of a silicone mould. A cast anhydrous product can, for example, comprise from 10% to 15% by weight of wax(es), from 0% to 10% by weight of paste(s) and from 60% to 70% by weight of oil(s).

The hardness of the inclusion elements—preferably when they are cast and moulded anhydrous products—is greater than the hardness of the fixing composition. For example, the hardness of the inclusion elements which are cast anhydrous products is greater than or equal to 2600 g, preferably between 2800 g and 4500 g. The inclusion part can have a greater hardness than that of the fixing composition.

The textures of moulded solid emulsion type used for the inclusion part are preferably water-in-oil emulsions.

The textures of powder type used for the inclusion part can be compacted by metal equipment without pots (which are known as powder cakes). Patterns can, in this case, be obtained with the compacting equipment. It is also possible to obtain patterns by compacting large surface areas of powder and by carrying out laser cutting of the chosen patterns. This process has the advantage that it avoids designing specific equipment for prototypes of patterns. Finally, the powders can be shaped in the form of slurries. The slurries are subsequently extruded and cut up using a hollow punch. The shapes obtained can be flat or slightly in relief.

Use may be made, as compacted powder, of a product comprising from 80% to 90% by weight of a mixture of powders which are chosen from fillers and pigments and from 10% to 20% by weight of oil(s) which is/are used as binder for the powders.

The cosmetic product of the invention can be a make-up product, a care product, a sun protection product or a scenting product.

The inclusion part and the fixing composition can be chosen independently from products for making up the lips, the eyes, cheeks, eyebrows, face and body, for example a lipstick, a blusher or an eye shadow.

In one embodiment, the fixing composition is an eye shadow, a blusher, a gloss or a lipstick. The inclusion part is, for example, a lipstick comprising waxes or an eye shadow in the form of pressed powders.

The inclusion part can comprise inorganic, organic or pearlescent pigments and optionally fillers. The fixing composition may or may not comprise pigments.

Mention may be made, among inorganic pigments, by way of examples, of titanium dioxide, optionally surface-treated; black, yellow, red and brown iron oxide; manganese violet; ultramarine blue, chromium oxide; chromium oxide hydrate and ferric blue.

Mention may be made, among organic pigments, for example, of the pigments D & C Red No. 19; D & C Red No. 9; D & C Red No. 21; D & C Orange No. 4; D & C Orange No. 5; D & C Red No. 27; D & C Red No. 13; D & C Red No. 7; D & C Red No. 6; D & C Yellow No. 5; D & C Red No. 36; D & C Orange No. 10; D & C Yellow No. 6; D & C Red No. 30; D & C Red No. 3; carbon black and lakes based on cochineal carmine.

The pearlescent pigments can be chosen in particular from white pearlescent pigments, such as mica covered with titanium oxide or bismuth oxychloride, and coloured pearlescent pigments, such as titanium oxide-coated mica with iron oxides, titanium oxide-coated mica with ferric blue or chromium oxide, or titanium oxide-coated mica with an organic pigment of the abovementioned type, and also pigments based on bismuth oxychloride.

The fillers can be chosen from talc, a magnesium silicate hydrate; micas having dimensions of 2 to 200 μm; kaolin, an aluminium silicate hydrate; zinc and titanium oxides; calcium carbonate, magnesium carbonate and basic magnesium carbonate; silica; zinc stearate, magnesium stearate, lithium stearate, zinc laurate or magnesium myristate; powders formed of synthetic polymers, such as polyethylene, polyesters and polyamides (for example nylon); spherical silica; spherical titanium dioxides; glass and ceramic beads; powders formed of organic materials of natural origin, such as maize, wheat or rice starches, which may or may not be crosslinked; powders formed of synthetic polymers which may or may not be crosslinked and which are spheronized, such as powders formed of polyamide, such as poly-β-alanine powders and nylon powders, powders formed of polyacrylic or polymethacrylic acids, polystyrene powders crosslinked by divinylbenzene, silicone resin powders or Teflon powders.

The drop point of the fixing composition is greater than 50° C., preferably greater than 55° C., for example between 55 and 60° C., between 70 and 80° C. or between 75 and 85° C.

The fixing composition preferably has a hardness of less than 2600 g. Its hardness is preferably greater than 600 g.

The fixing composition has, for example,
a hardness of between 1600 g and 2600 g and a drop point of between 70 and 80° C., or
a hardness of between 600 g and 1700 g and a drop point of between 75 and 85° C., or
a hardness of between 1000 g and 1600 g and a drop point of between 55 and 60° C.

The hardness of the compositions can be measured with a texture analyser from Stable Micro Systems, model TA.XT.Plus, by penetration into the composition of a probe, for example a hemispheric probe, at a rate and a force assigned by the apparatus.

The drop point can be measured with a Mettler Toledo DP70 apparatus provided with standardized cups having an orifice diameter of 2.8 mm by adopting a heat rate of 2.0° C./min, a starting temperature of 40° C. and an end temperature of 95° C. The drop is detected visually using a camera incorporated in the apparatus.

The fixing composition comprises a structuring compound which can be chosen from waxes and oil-gelling polymers. When the fixing composition comprises waxes, it is preferable for it to comprise not very much of them so that its hardness is not excessively high. Generally, the incorporation of more than 10% by weight of wax(es) in the composition will be avoided. The choice may be made to incorporate pasty materials in order to structure the fixing composition without increasing its hardness.

The fixing composition is, for example, an anhydrous gel comprising an oil and an agent which gels this oil, such as described in the document FR 2 958 159 or FR 2 975 589. Such a gelling agent is, for example, chosen from polyamides, such as polyamide copolymers comprising an ester ending (Uniclear®, Union Carbide), or else silicone polyamides, or else L-glutamide derivatives, such as dibutyl lauroyl glutamide, sold by Ajinomoto, or one of their mixtures, fumed silicas, or else hydrogenated or nonhydrogenated copolymers comprising at least one styrene unit, such as, for example, a commercially available hydrogenated styrene/methylstyrene/indene copolymer.

According to one embodiment, the gelling agent is a polymer of polyamide type comprising an amide ending, preferably a polymer of polyamide type comprising a tertiary amide ending (ATPA), of formula (I) below:

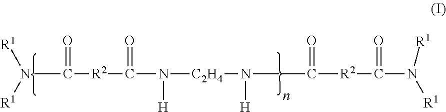

in which:
n is an integer denoting the number of units present in the formula (I), it being possible for n to vary from 1 to 10, preferably from 1 to 5,
the R¹ radicals, which are identical or different, denote a hydrocarbon group comprising from 1 to 22 carbon atoms,
the R² radicals, which are identical or different in each instance, denote a hydrocarbon group comprising from 2 to 54 carbon atoms, with the proviso that at least 50% of the R² radicals have from 30 to 42 carbon atoms.

A first preferred polymer of ATPA type is a copolymer having the name (INCI) Ethylenediamine/Hydrogenated Dimer Dilinoleate Copolymer bis-di-C₁₄₋₁₈ Alkyl Amide, which is commercially available.

A second preferred polymer of ATPA type is a copolymer having the name (INCI) Bis-Dioctadecylamide Dimer Dilinoleic Acid/Ethylenediamine Copolymer, which is commercially available.

The composition according to the invention advantageously comprises from 10% to 50% by weight of the composition, more preferably from 15% to 30% by weight of the composition, of a polymer of ATPA type.

Use may be made, as oil, of the mixture of non-volatile hydrogenated polyalkylene oil, in particular a hydrogenated polyisobutene, and of a fatty acid ester comprising at least one free hydroxyl group, for example a hydroxystearate ester, preferably ethylhexyl hydroxystearate, when the gelling agent is an ATPA.

In a specific embodiment, the composition advantageously comprises:
from 15% to 30% by weight of a polymer of ATPA type having the name (INCI) Bis-Dioctadecylamide Dimer Dilinoleic Acid/Ethylenediamine Copolymer,
from 40% to 80% by weight of hydrogenated polyisobutene,
from 5% to 20% by weight of ethylhexyl hydroxystearate and, optionally:
from 0% to 5% by weight of cetyl alcohol,
from 0% to 5% of a dibutyl lauroyl glutamide mixture,
from 0% to 15% of a hydrogenated styrene/methylstyrene/indene copolymer.

The fixing composition can comprise, for example, a mixture of oil(s), wax(es) and paste(s). For example, the oils represent between 55% and 65% by weight of the composition, while the waxes represent from 2% to 10% by weight of the composition and the pastes represent from 20% to 30% by weight of the composition.

The oils are, for example chosen from esters, polybutene, polydecene and octyldodecanol. The pastes are, for example a polyisobutene, a C₁₀-C₃₉ ester, an olefin copolymer or one of their mixtures. Use may be made, as wax, of polyethylene wax, a beeswax, an ozokerite wax or one of their mixtures.

The fixing composition can be anhydrous or can comprise both an aqueous phase and a fatty phase. The fixing composition can be, for example, a water-in-oil emulsion which is solid at ambient temperature.

The product of the invention can comprise the assembly of an anhydrous fixing composition and anhydrous inclusion parts, of an anhydrous fixing composition and inclusion parts as a water-in-oil emulsion, of a fixing composition as a water-in-oil emulsion and anhydrous inclusion parts, or of a fixing composition as a water-in-oil emulsion and inclusion parts as a water-in-oil emulsion.

In a first specific embodiment, the product of the invention comprises the assembly of a fixing composition in the form of an anhydrous gel comprising Bis-Dioctadecylamide Dimer Dilinoleic Acid/Ethylenediamine Copolymer and hydrogenated polyisobutene, and anhydrous inclusion parts comprising more than 70% by weight of powders.

In a specific embodiment, the product of the invention comprises the assembly of a fixing composition comprising at least one pasty fatty compound, oils and less than 10% by weight of waxes, and anhydrous inclusion parts comprising more than 10% by weight of waxes and more than 50% by weight of oils.

The product of the invention can comprise at least one applicator, in the form of a brush, including a fine brush, or foam tip fixed to a rod. The applicator can be housed in and snap into the container.

According to a second aspect, the invention relates to a process for the manufacture of a cosmetic product as described above which comprises the series of following stages:
shaping of the inclusion part,
positioning the inclusion part in the cavity of the container,
heating the fixing composition to a temperature greater than its melting point and casting the said composition in the spaces of the cavity left empty by the inclusion part,
cooling the fixing composition until it has solidified.

Once the fixing composition has been poured into the container, it has a surface in contact with the container, a surface in contact with the inclusion part and a free surface. The free surface of the fixing composition is preferably flat, whereas the surface of the inclusion part from which the user can remove the product is higher than the free surface of the fixing composition. The term “higher” is understood to mean higher with respect to the bottom of the container, with the result that the inclusion part projects with respect to the fixing composition.

The inclusion part is generally placed on the bottom of the container without coming into contact with the edges of the latter, so that the space left free between the inclusion part and the edges of the container is filled with the fixing composition. In the product of the invention, the fixing composition can thus surround the base of the inclusion part so as to immobilize it with respect to the container.

In the case of a cast anhydrous product, the shaping of the inclusion part can be carried out by the series of following stages: melting a mixture predominantly comprising a fatty phase which is solid at 25° C., casting the said mixture in a silicone mould, cooling the said mixture and then removing the part thus formed from the mould.

Silicone moulds can be used in order to obtain different shapes and reliefs. By using silicone moulds, cast cosmetic inclusion products can be in relief, the shapes are pronounced in three dimensions and the patterns are subtle, which renders the product more attractive.

In the case of a pressed powder, the shaping of the inclusion part can be carried out by the series of following stages: compacting, with the help of an impression, a mixture comprising a predominant proportion of powders, in order to form pot-free powder cakes.

In the case of a pressed powder, the shaping of the inclusion part can also be carried out by the series of following stages: compacting a mixture comprising a predominant proportion of powders and laser cutting according to the desired shape.

The shaping of the inclusion part can also be carried out by the series of following stages: mixing powders in a solvent in order to obtain a paste which is extruded, cutting the paste with a hollow punch according to the desired shape, and drying.

According to the process of the invention, the solidification of the surface of the fixing composition is advantageously carried out in a few seconds to a few minutes at ambient temperature (25° C.).

The fixing composition can be cast at a temperature of less than or equal to 99° C., more particularly less than 95° C.

In one embodiment, the container has a temperature before the casting of the fixing composition which is equal to the casting temperature of the fixing composition. Alternatively, the container has a temperature before the casting of the fixing composition which is less than the casting temperature of the fixing composition, for example of the order of 20 to 25° C.

The container can be a packaging device in the form of a case comprising a bottom defining a first hollow compartment for receiving the assembly of the inclusion part and the fixing composition. The case comprises a lid which can be articulated on the bottom and comprise a recess in the bottom of which is fitted a mirror. It can be made of plastic or of metal.

The bottom can define a recess in which is positioned at least one applicator, such as a foam or a fine brush.

In the cosmetic product of the invention, just one compartment comprises several different compositions. On the contrary, in the prior art, the bottom of the case defines several compartments each intended to receive a different product and pots or dishes are positioned in each of the compartments, which pots or dishes will be stuck or fitted into the compartments, with the result that the cosmetic compositions are not cast or compacted directly in the case.

The invention does not relate in particular to products in the form of bicoloured lipsticks or foundation sticks which do not comprise a flat-bottomed container. In these products, the hardnesses of the two colours are equivalent in order to guarantee deposition of the product on the skin without crushing the cast or premature wear of one colour with respect to the other.

### DETAILED DESCRIPTION OF THE FIGURES

FIG. 3 represents a view in perspective of a cosmetic make-up product (1a) according to the invention. In FIGS. 1 and 2, three spherical inclusion elements (4a) which have been moulded and removed from the mould beforehand are placed on the flat bottom of the circular cavity (3a) of a container (2a) of square shape. These inclusion elements predominantly comprise fatty substances comprising a mixture of oils and waxes. In FIG. 3, the inclusion elements have been assembled with one another and rendered integral with the container by casting, in the cavity (3a), a fixing composition (5a) preheated to 95° C. The fixing composition, once cast, solidifies in a few minutes, which makes it possible to handle the product very rapidly. The fixing composition comprises waxes, oils and pastes; its hardness is less than 2800 g and its drop point is greater than 50° C. The relative size of the inclusion elements and the height of the cavity are chosen so that the inclusion parts show on the surface of the fixing composition which fills the cavity and produce a cosmetic product in relief.

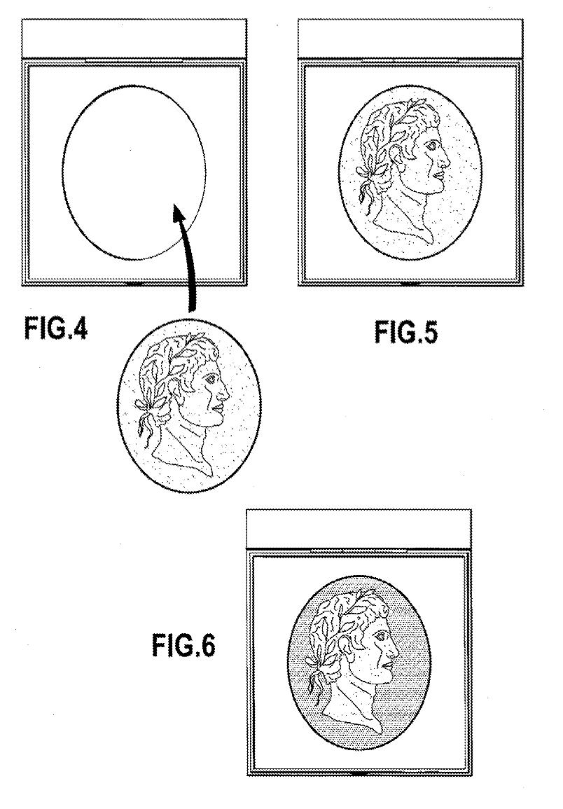

FIG. 6 represents a view in perspective of a scenting cosmetic product (1b) according to the invention. In FIGS. 4 and 5, an inclusion element in the form of a bust (4b) which has been moulded and removed from the mould beforehand is placed on the flat bottom of the oval cavity (3b) of a container (2b) of square shape. In FIG. 6, the bust has been rendered integral with the container by casting a fixing composition (5b) at 95° C. so as to fill the cavity (3b).

FIG. 9 represents a view in perspective of a cosmetic make-up product (1c) according to the invention. In FIGS. 7 and 8, four spherical inclusion elements (4c) which have been shaped beforehand by compacting are placed on the flat bottom of the square cavity (3c) of a container (2c) of square shape. These inclusion elements predominantly comprise powders which have been pressed. In FIG. 9, the inclusion elements have been assembled with one another and rendered integral with the container by casting, in the cavity (3c), a fixing composition (5c) preheated to 95° C. The fixing composition, once cast, solidifies in a few minutes, which makes it possible to handle the product very rapidly. The fixing composition is a pearlescent transparent gel comprising oils gelled by a polymer; its hardness is less than 2800 g and its drop point is greater than 50° C. The relative size of the inclusion elements and the height of the cavity are chosen so that the inclusion parts show on the surface of the fixing composition which fills the cavity and produce a cosmetic product in relief.

The invention is illustrated by the following examples.

### Example

### Assemblies of Inclusion Elements and a Cast Fixing Composition

Each product is composed of at least one inclusion element which is deposited at the bottom of a cavity formed in a unitary packaging, in which cavity a fixing composition is cast. All of the elements are formed of cosmetic ingredients, the complete combination forming a composition for making up, caring for or protecting the skin, in particular the skin of the face.

The hardness of the compositions was measured with a texture analyser from Stable Micro Systems, model TA.XT.Plus, by penetration of a hemispheric probe having a diameter of 12.7 mm (known as Delrin finger). The measuring was carried out in a 100 ml Cleopatra pot and it was repeated three times.

The parameters of the measurement were as follows:
Approach 2 mm/s
Test 2 mm/s
Withdrawal 2 mm/s
Penetration 13 mm
Relaxation 25 sec
Triggering force: 2 g.

The drop point was measured with a Mettler Toledo DP70 apparatus provided with standardized cups having an orifice diameter of 2.8 mm by adopting a heat rate of 2.0° C./min, a starting temperature of 40° C. and an end temperature of 95° C. The drop is detected visually using a camera incorporated in the apparatus.

### 2.1 Inclusion Elements of Composition A

Composition A is used for the preparation of inclusion elements of cast type. The cosmetic “inclusion” elements of cast type are, for example, shaped by casting in silicone moulds.

Composition A exhibits the following formulation (% by weight):

| | % |
|---|---|
| Waxes | 12.7 |
| Pastes | 3.4 |
| Oils | 64.5 |
| Fillers | 15.9 |
| Others | q.s. for 100 |

The waxes are a mixture of polyethylene, beeswax and ozokerite. The pastes are composed of a mixture of Bentone and C₁₀-C₃₀ esters. The oils are composed of a mixture of polyisobutene, esters, polydecene and octyldodecanol. The fillers are composed of pigments and pearlescent agents, the respective natures and proportions of which vary as a function of the colour which it is desired to give to the composition. The part “Others” is composed of preservatives, active agents or fragrances.

Composition A exhibits a drop point of 78.2° C., a melting point of 77.3° C. and a mean hardness of 4424 g, measured according to the methods described above.

### 2.2 Inclusion Elements of Composition B

Composition B is used for the preparation of inclusion elements. Cosmetic “inclusion” elements of dry powder type are shaped by a conventional process, such as by compacting or by extrusion, cutting with a hollow punch and drying.

A composition of eye shadow type is prepared which has the following formulation (% by weight):

| | % |
|---|---|
| Dimethicone-treated talc | 29.4 |
| Magnesium Stearate | 2.0 |
| Nylon 12 | 3.0 |
| Calcium Sodium Borosilicate | 0.5 |
| Pigments | 0.4 |
| Pearlescent agents | 50.0 |
| Esters | 14.0 |
| Phenoxyethanol | 0.7 |

The same formulation is reproduced by varying the composition in pigment so as to obtain a pink, brown or beige colour.

### 2.3 Fixing Composition C

The fixing composition is prepared in the form of a transparent gel. This gel can be used itself as fixing composition or else can be additivated with a filler comprising pigments and/or pearlescent agents.

The formulation of the transparent gel is described in detail below (% by weight):

| | % |
|---|---|
| Phase A | |
| Bis-Dioctadecylamide Dimer Dilinoleic | 21 |
| Acid/Ethylenediamine Copolymer | |
| Ethylhexyl hydroxystearate | 11 |
| Hydrogenated polyisobutene | 56 |
| Phase B | |
| Hydrogenated styrene/methylstyrene/indene copolymer | 10 |
| Cetyl Alcohol | 3.2 |
| Dibutyl Lauroyl Glutamide | 0.4 |
| Dibutyl Ethylhexanoyl Glutamide | 0.4 |

### Process for the Preparation of the Gel

The various components of phase A are melted at 95° C. with stirring (Rayneri TurboTest, 300 revolutions/mn). Phase B is added and melting is allowed to take place with stirring.

8% of pearlescent agents and 92% of the preparation obtained above are dispersed. The pearlescent agents are dispersed homogeneously in the A+B molten phase.

Composition C is a coloured gel exhibiting a hardness of 654 g according to the method described above and a drop point equal to 82.8° C.

### 2.4 Fixing Composition D

Use is made, as fixing product, of a composition D having the formulation (% by weight):

| | % |
|---|---|
| Waxes | 10.2 |
| Pastes | 25.5 |
| Oils | 60.3 |
| Fillers | 0.7 |
| Others | q.s. for 100 |

The waxes are a mixture of polyethylene, paraffinic wax and ozokerite wax. The pastes are composed of a mixture of EPS copolymer, Bentone and hydrogenated polyisobutene. The oils are composed of a mixture of polybutene, esters and octyldodecanol. The fillers are composed of pigments and pearlescent agents, the respective natures and proportions of which vary as a function of the colour which it is desired to give to the composition. The part “Others” is composed of preservatives, active agents or fragrances.

The composition exhibits a drop point of 75.4° C. and a mean hardness of 1867 g, measured according to the methods described above.

### 2.5 Fixing Composition E

A fixing composition is prepared in the form of a solid water-in-oil emulsion having the following formulation. The names of the ingredients correspond to their chemical name or to their INCI name (% by weight).

| | % |
|---|---|
| Polyethylene wax | 3 |
| Beeswax | 3 |
| Hydrogenated glycerides | 3.7 |
| Hectorite | 0.8 |
| Esters | 5.45 |
| Oils | 9.7 |
| Liquid sunscreens | 7.5 |
| Pigments | 19 |
| Water | q.s. for 100 |
| Emollients and preservatives | 5.6 |
| Vinyl dimethicone/methicone silsesquioxane crosspolymer, | 4.6 |
| cellulose gum and polymethylsilsesquioxane | |
| Solid sunscreens | 1.5 |

The fatty phase is prepared by melting the waxes and by then adding, at 85° C., the pastes and a portion of the oils at 85° C. The hectorite is subsequently dispersed, followed by the pigments, dispersed beforehand in the remainder of the oils.

The aqueous phase is prepared by heating the water and the emollients to 85° C. and then the vinyl dimethicone/methicone silsesquioxane crosspolymer, cellulose gum and polymethylsilsesquioxane mixture is dispersed therein.

The aqueous phase is poured into the fatty phase at 85° C. and the mixture is emulsified for 15 min. The preservatives are added.

The hardness and the drop point are measured according to the methods described above:
Hardness: 1302 g
Drop point: 58.4° C.

The emulsion obtained is used as fixing agent for the moulded elements. The emulsion is poured into the container in which the moulded objects have been placed beforehand.

### 2.6 Fixing Composition Ca

A fixing composition of the invention was prepared by using the following ingredients.

| Phase A | |
|---|---|
| Bis-Dioctadecylamide Dimer Dilinoleic Acid/ | 16.5 |
| Ethylenediamine Copolymer | |
| Ethylhexyl Hydroxystearate | 12 |
| Hydrogenated polyisobutene | 60.5 |
| Phase B | |
| Hydrogenated styrene/methylstyrene/indene copolymer | 7.0 |
| Cetyl Alcohol | 3.2 |
| Dibutyl Lauroyl Glutamide | 0.4 |
| Dibutyl Ethylhexanoyl Glutamide | 0.4 |

| | Ca |
|---|---|
| Hardness | 1643 g |
| Drop point | 83.4° C. |

### 2.7 Assembly According to the Invention of Inclusion Elements and Fixing Compositions

The inclusion elements were placed on the bottom of a case or a dish and then the fixing product, brought beforehand to a temperature of 95° C., was cast in the space of the compartment or of the dish left free by the inclusion elements. The fixing composition was subsequently allowed to cool.

The following assemblies were produced.

| | Inclusion | Fixing | |
|---|---|---|---|
| Assembly | elements | composition | Observation |
| 1 | Composition A | Composition D | FIG. 3 |
| 2 | Composition B | Composition C | FIG. 9 |
| 3 | Composition A | Composition E | FIG. 6 |
| 4 | Composition A | Composition Ca | — |

The compositions obtained are solid and are used for making up the skin.

Use is made without distinction of compositions A and B to prepare inclusion parts and compositions C, D, E and Ca as fixing products to prepare cosmetic compositions, the use of which can be the making up or else the care of the skin or else again the protection of the skin when the compositions comprise, for example, screening agents which confer an SPF value on them.

Composition Ca has a drop point greater than the drop point of the inclusions A. In point of fact, it is observed that, cast hot at 95° C., the composition Ca indeed uniformly occupies the bottom of the container, indeed surrounds the inclusions and completely fills the small-size interstices, in particular those of FIG. 5. Furthermore, although the casting temperature is greater than the melting point of the inclusions A, the shapes and the delimitations of the latter remain intact. This runs counter to the teaching of U.S. Pat. No. 6,058,942.

1. In relief cosmetic product comprising a container having at least one cavity in which is positioned an assembly of at least one inclusion part which is solid at 25° C. and of a fixing composition which is solid at 25° C., characterized in that:
the inclusion part and the fixing composition are in contact with one another and have different compositions,
the fixing composition has a drop point greater than 50° C. and a mean hardness, measured at 25° C. using a texture analyser provided with a hemispheric probe, of less than 2800 g, and
the fixing composition comprises at least one oil and one structuring compound.

2. Cosmetic product according to claim 1, characterized in that the container comprises just one cavity and in that it is pot-free.

3. Cosmetic product according to claim 1, characterized in that it comprises at least two inclusion parts.

4. Cosmetic product according to claim 1, characterized in that the said product is a make-up product, a care product, a sun protection product or a scenting product.

5. Cosmetic product according to claim 1, characterized in that the fixing composition is an eye shadow, a blusher, a gloss or a lipstick.

6. Cosmetic product according to claim 1, characterized in that the hardness of the inclusion part is greater than the hardness of the fixing composition.

7. Cosmetic product according to claim 1, characterized in that the structuring compound is chosen from waxes and oil-gelling polymers.

8. Cosmetic product according to claim 1, characterized in that the inclusion part is a lipstick comprising waxes or an eye shadow in the form of pressed powders.

9. Cosmetic product according to claim 1, characterized in that the inclusion part projects with respect to the fixing composition.

10. Cosmetic product according to claim 1, characterized in that the fixing composition has a hardness of between 1600 g and 2600 g and a drop point of between 70 and 80° C.

11. Cosmetic product according to claim 1, characterized in that the fixing composition has a hardness of between 600 g and 1700 g and a drop point of between 75 and 85° C.

12. Cosmetic product according to claim 1, characterized in that the fixing composition has a hardness of between 1000 g and 1600 g and a drop point of between 55 and 60° C.

13. Process for the manufacture of a cosmetic product according to claim 1, characterized in that it comprises the series of following stages:
shaping of the inclusion part,
positioning the inclusion part in the cavity of the container,
heating the fixing composition to a temperature greater than its melting point and casting the said composition in the spaces of the cavity left empty by the inclusion part,
cooling the fixing composition until it has solidified.

14. Process according to claim 13, characterized in that the shaping of the inclusion part is carried out by the series of following stages: melting a mixture predominantly comprising a fatty phase which is solid at 25° C., casting the said mixture in a silicone mould, cooling the said mixture and then removing the part thus formed from the mould.

15. Process according to claim 13, characterized in that the shaping of the inclusion part is carried out by the series of following stages: compacting, with the help of an impression, a mixture comprising a predominant proportion of powders, in order to form pot-free powder cakes.

16. Process according to claim 13, characterized in that the shaping of the inclusion part is carried out by the series of following stages: compacting a mixture comprising a predominant proportion of powders and laser cutting according to the desired shape.

17. Process according to claim 13, characterized in that the shaping of the inclusion part is carried out by the series of following stages: mixing powders in a solvent in order to obtain a paste which is extruded, cutting the paste with a hollow punch according to the desired shape, and drying.

18. Process according to claim 13, characterized in that the solidification of the surface of the fixing composition is carried out in a few seconds to a few minutes at 25° C.

19. Process according to claim 13, characterized in that the fixing composition is cast at a temperature of less than or equal to 99° C., more particularly of less than 95° C.

20. Process according to claim 13, characterized in that the container has a temperature before the casting of the fixing composition which is less than the casting temperature of the fixing composition.

21. Process according to claim 13, characterized in that the container has a temperature before the casting of the fixing composition of the order of 20 to 25° C.
